(19) United States
(12) Patent Application Publication
(10) Pub. No.: US 2008/0319232 A1
(43) Pub. Date: Dec. 25, 2008

(54) Synthesis of Aldehydes by Ozonolysis of Secondary Allyl

(75) Inventors: Markus Nobis, Lyss (CH); Bernd Hoelscher, Halle (DE)

Correspondence Address: CONNOLLY BOVE LODGE & HUTZ, LLP, P O BOX 2207, WILMINGTON, DE, 19899, US

(73) Assignee: SYMRISE GmbH & Co. KG, Holzminden (DE)

(21) Appl. No.: 12/088,308

(22) PCT Filed: Aug. 3, 2006
(86) PCT No.: PCT/EP2006/065053

§ 371 Date: Aug. 26, 2008

(30) Foreign Application Priority Data
Sep. 28, 2005 (DE) 10 2005 046 535.8

(51) Int. Cl. C07C 45/40

(52) U.S. Cl. 568/469

(57) ABSTRACT

The invention relates to a process for the preparation of an aldehyde from a secondary alcohol having a double bond in the alpha position, comprising the steps.
a) provision of the alcohol,
b) treatment of the alcohol with ozone.

The invention relates to a process for the preparation of aldehydes from secondary alcohols having a double bond in the alpha position (in the following also: secondary allyl alcohols) by means of ozonolysis.

Tertiary alcohols having a double bond in the alpha position are conventionally converted into the corresponding carbonyl compounds in the presence of inorganic oxidizing agents (e.g. KMnO₄, OsO₄, H₂SO₄/H₂CrO₄). In particular, it is known to convert an alcohol of the formula (I) into a ketone of the formula (II) in this manner:

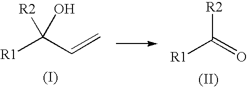

wherein the radicals R1, R2 independently of one another can be alkyl, alkenyl, alkynyl, cycloalkyl or aryl. In this context, the radicals can be branched or unbranched, substituted or unsubstituted, and the aryl radicals can furthermore be fused. Alcohols of the formula (I) are obtained from natural sources in some cases, but are also accessible by synthesis.

On the other hand, the preparation of aldehydes from secondary alcohols having a double bond in the alpha position (R2=H in formula (I)) by this route proves to be problematic. Because of the reaction mechanism, by-products are formed due to the various possibilities of fragmentation of the intermediates, as shown in the following equation:

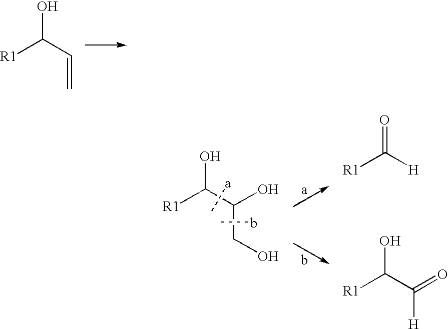

Aldehydes are important (intermediate) products for various branches of the chemical industry. Aldehydes can be prepared, for example, by reaction of olefinic compounds with a mixture of carbon monoxide and hydrogen on metal catalysts (hydroformylation), by reaction of aromatics with dimethylformamide (so-called Vilsmeier-Haak synthesis), or also by the Darzens reaction on ketones.

The preparation methods mentioned are distinguished by the use of metal-containing reactants or by the use of toxic reagents. The particular methods are furthermore limited to the particular substrate class and the existence of certain characteristics in the molecule.

Various examples in which ketones are formed by ozonolytic degradation of tertiary allyl alcohols are known in the literature. WO 91/09852 describes, for example, a two-stage process for the preparation of sclareolide (also (−)-norlabdan oxide) from sclareol, in which in a first stage an oxidative degradation of sclareol is carried out in the presence of ruthenium salts or potassium permanganate, and in a second stage the intermediate product formed is oxidized with peracid and/or peracid salts to give sclareolide.

The oxidizing agents used in conventional processes are a disadvantage because of their toxicity to man and the environment and their ease of handling being made difficult as a result. This disadvantage in particular makes the industrial reaction of secondary alcohols having a double bond in the alpha position difficult.

Attempts have therefore been made to modify these processes, and in particular to use novel oxidizing agents. By the example of the reaction of sclareol, EP 0 822 191 A1 and Fekih et al., (J. Soc. Chim. Tunisie, 2001, 4(9), 909) have described the two-stage process in each case for the preparation of sclareol oxide (VIIb) from the tertiary allyl alcohol sclareol (VI) by ozonolysis:

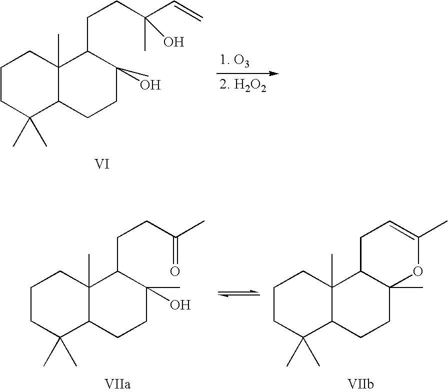

In a first stage, the allyl alcohol group of the sclareol is converted into the corresponding ozonide by addition of ozone. In a second stage, the ozonide is then converted into the desired sclareol oxide by working up with alkaline H₂O₂. The reaction can be carried out in various organic solvents, such as methylene chloride, methanol or ethanol.

In the reaction, however, large amounts of the highly reactive ozonide are obtained in the first step, so that considerable safety precautions are necessary for carrying out the reaction. In particular, an efficient cooling is required in order to be able to carry out the reaction safely. These disadvantages are important in particular in an industrial reaction.

The invention is based on the object of providing a process for the preparation of an aldehyde from a secondary alcohol having a double bond in the alpha position which limits or completely avoids the abovementioned disadvantages of conventional oxidation processes. In particular, it should be possible to carry out the process without the high safety precautions hitherto necessary. Likewise, the use of metal-containing and/or toxic oxidizing agents is to be avoided and the formation of aldehydes is to be rendered possible, without further oxidation to the corresponding carboxylic acids taking place to a noticeable extent. The object is achieved by a process for the preparation of an aldehyde from a secondary allyl alcohol, comprising the steps:

a) provision of the alcohol,
b) treatment of the alcohol with ozone.

The process according to the invention leads in a surprisingly short reaction time to the desired reaction products in a simultaneously high yield.

It is preferable to allow the treatment of the alcohol with ozone in step b) to take place in the presence of an inorganic base. Surprisingly, a very selective formation of the aldehyde, in spite of the basic reaction conditions, can be observed, although in general aldehydes tend to form the corresponding aldol condensation products under basic reaction conditions.

The possibility of achieving high yields in a simultaneously short reaction time was furthermore surprising in particular since it was known that in the presence of an inorganic base, ozone dissociates rapidly (especially at weakly basic pH values) (Hollemann, Wiberg, Lehrbuch der Anorganischen Chemie [Textbook of Inorganic Chemistry], W. de Gruyter 1995, 101st ed., p. 516). Accordingly, it was to be expected that large amounts of ozone would be required in order to provide a sufficient amount of ozone for reaction of the secondary allyl alcohol. It has now been found, surprisingly, that the amount of ozone required is not increased compared with conventional processes, in spite of the presence of an inorganic base, and that the reaction according to the invention, which is preferably carried out in one step, can even be carried out significantly more rapidly and with a lower requirement of safety precautions in the presence of an inorganic base than in the case of conventional processes. It has likewise been possible to demonstrate that by reaction of the secondary allyl alcohol with ozone under the conditions mentioned, no or no noticeable further oxidation to the corresponding carboxylic acid is to be observed. This can be achieved only in part under the conditions of oxidation with metal-containing inorganic compounds.

This variant of ozonolysis furthermore opens up the possibility of generating molecules with formyl groups from a halide and acrolein (propenal), the starting molecule being lengthened by one carbon atom by this reaction sequence, R1 in the following also having the meaning given above.

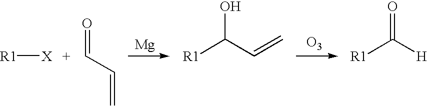

In addition to hydroformylation, this thus represents a more diverse method than the methods in the literature used to date. Possible halides R1-X are chlorides, bromides and iodides. The addition of the carbon radical on to the propenal is carried out here by the conventional methods known from the literature (Organikum, 16th ed., p. 495 et seq., VEB Deutscher Verlag der Wissenschaften, 1985).

It is likewise possible to provide an industrially usable method for introduction and removal of protective groups by a reaction sequence comprising nucleophilic addition of a vinyl group (for example by means of vinyllithium or a vinylmagnesium halide) on to an aldehyde and by subsequent ozonolysis:

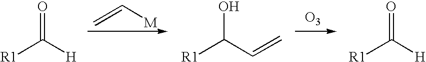

M preferably denotes MgCl, MgBr, MgI or Li.

A further advantage of the process according to the invention is that in the procedure, in particular in the presence of an inorganic base during step b), only small amounts of heat are released. Compared with conventional processes, only cooling units having a lower output are therefore required for carrying out the process according to the invention. This is a great advantage in particular in an industrial process procedure.

Particularly good results can be obtained if, in step b), the alcohol employed is treated with 1-3 molar equivalents of ozone, based on the alcohol group to be reacted. In this context, a process according to the invention in which in step b) the alcohol employed is treated with 1-2 molar equivalents of ozone, based on the alcohol group to be reacted, is particularly preferred. In both cases, the amount of ozone employed is advantageously kept low. This is of advantage in particular in an industrial process procedure, since ozone is expediently generated in a reaction which runs in parallel during the reaction of the alcohol and is added constantly or continuously to the reaction which proceeds in step b). The process according to the invention therefore renders possible a reaction of the alcohol with a low requirement of ozone to be provided.

For generation of the ozone in an ozone generator, pure oxygen, but also mixtures of oxygen and inert gases in various volume ratios of oxygen, preferably between 1 and 80 vol. %, can be used. An ozone content of a gas passed into the reaction mixture in step b) is preferably in the range of from 1 to 12 wt. %, based on the gas employed, but particularly preferably in the range of from 4 to 8 wt. %. The ozone can be passed into the reaction mixture in a molar amount in the range of from 1 to 5, preferably in the range of from 1 to 3, particularly preferably in the range of from 1.1 to 2 molar equivalents to the double bond to be reacted in the alpha position of the compound. By-products of the ozonolysis can be decreased by this means.

It is furthermore preferable if, in step b), the base is not already initially introduced completely at the start of the reaction, but is added constantly such that its equivalent concentration on discontinuation of the reaction, based on the total alcohol groups to be reacted which are employed, is 1 to 3, preferably 1 to 2. It is ensured in this way that the concentration of available ozone is at the optimum level to achieve a rapid reaction of the alcohol with high yields, and at the same time is low enough to prevent the release of high amounts of heat in step b). The base can be added continuously or repeatedly when carrying out step b).

Suitable inorganic bases are all the strong to medium-strong Brönstedt bases which are stable under ozonolysis conditions. The base used in step b) is preferably chosen from the group consisting of NaOH, KOH, LiOH, NaHCO₃, Na₂CO₃, CaCO₃ or mixtures of two or more of these bases. In this context, the alkali metal bases mentioned are in turn advantageous, and the alkali metal hydroxides are preferred. Particularly preferred bases are NaOH, KOH and LiOH, and NaOH and KOH are most preferred. In the process according to the invention, with alkali metal bases no corresponding alkali metal peroxides or alkali metal ozonides are formed or accumulated, in contrast to the process described in U.S. Pat. No. 3,664,810 with alkaline earth metal bases, in which substantially stoichiometric amounts of the corresponding alkaline earth metal peroxides are formed. These bases, in particular the alkali metal hydroxides (which are mentioned as particularly preferred) have delivered particularly high yields of the desired compound in comparison experiments. The bases are expediently provided in dissolved form in step b), so that when choosing the base, the solubility thereof in the solvent used is also to be taken into account.

EP 1 569 885 relates to the in situ dissociation of peroxides during the ozonolysis of optionally substituted alkenes to give the corresponding aldehydes or ketones. CaCO₃, inter alia, can be used as the support material for the peroxide-dissociating metal catalysts used there. According to EP 1 569 885, CaCO₃ is not employed for its basic properties, but, on the contrary, as an inert carrier material which does not dissolve in the solvent (mixture) employed in the ozonolysis and therefore also makes no noticeable contribution to the pH.

The process according to the invention is preferably carried out at a pH in the range of from 13 to 8. The pH is regularly initially in the range of from 13 to 12 during the ozonolysis in the presence of the base in step b) of the process according to the invention, and in the range of from 9 to 8 at the end of the ozonolysis. In this context, it is to be noted that the selectivity of the ozonolysis in is the process according to the invention does not decrease as the duration of the reaction progresses. This is all the more surprising, since it is known that the stability of ozone decreases noticeably at lower pH values (below 14) (Hollemann, Wiberg, Lehrbuch der Anorganischen Chemie [Textbook of Inorganic Chemistry], W. de Gruyter 1995, 101st ed., p. 508, 517). It would have been expected that the dissociation products of ozone would have reacted with the tertiary alcohols having a double bond in the alpha position which are to be employed according to the invention, to give undesirable by-products or degradation products of the tertiary alcohols.

In preferred embodiments, the process according to the invention is carried out in the absence of a heterogeneous, inorganic peroxide-dissociating catalyst from the group consisting of iridium, manganese, cobalt, silver, gold, palladium, platinum or ruthenium.

In further preferred embodiments, the process according to the invention is carried out in the absence of an emulsifier.

Water or a solvent mixture of water and a water-miscible organic solvent is preferably employed as the solvent for the base in step b). The solvent mixture preferably comprises tetrahydrofuran and water, in particular with a mixture ratio by weight of tetrahydrofuran to water in the range of from 1:2 to 2:1, particularly preferably about 1:1. The solvent must be suitable for the ozonolysis. The base is preferably added in step b) by dropwise addition from a stock solution, the concentration of the base in the stock solution preferably being 2 to 50 wt. %, particularly preferably 7.5 to 10 wt. %, in each case based on the total stock solution.

The solvent for the alcohol is chosen such that it is completely or largely inert towards ozone and is completely or largely stable towards the base added. Preferred solvents for the alcohol include substituted or unsubstituted aromatic hydrocarbons, or solvents which contain oxygen in the form of carbonyl, ether or alcohol functionalities. Halogenated aromatic and non-aromatic solvents likewise is prove to be suitable for carrying out the reaction. Solvents with other oxidizable hetero atoms (nitrogen and sulfur) are not very suitable or unsuitable because of their affinity for oxygen. Toluene is particularly preferred.

It is particularly preferable for the reaction in step b) to be carried out in a two-phase system, the alcohol being provided in an organic solvent in step a) and the base being employed in an aqueous solvent in step b). This has the advantage that precipitation of the base on addition into the reaction mixture is prevented, a concentration of the base in the phase of the reaction mixture containing the alcohol remains low, and a reaction between the ozonide formed and the base takes place only in the region of the phase boundary. The suppression of parallel and consecutive reactions (e.g. aldol reaction) by the use of a multi-phase system is likewise advantageous. In this context, the reaction mixture is expediently mixed thoroughly by stirring. Particularly preferably, the solvent of the alcohol is toluene and the solvent of the base is water or a solvent mixture of water and tetrahydrofuran, in particular with a mixture ratio by weight of tetrahydrofuran to water in the range of from 1:2 to 2:1, particularly preferably about 1:1.

Preferably, the base in step b) is added to the reaction mixture with a rate of addition which depends on the amount of ozonide formed. As a rule, the rate of addition of the base is increased when the amount of ozonide formed also increases during the period in question, and vice versa. It is particularly preferable for the dissolved base to be added in a molar amount of between 0.8 and 1.2 molar equivalents to the ozonide formed. By this means, the concentration of the ozonide in the reaction mixture can be kept low, but possible side reactions or disturbances in the formation of the ozonide due to the base added are avoided.

Preferably, in step b) the reaction temperature is −78° C. to +30° C., in particular −30° C. to +10° C., particularly preferably −10° C. to 0° C. By this means, side reactions of the ozonolysis and during the further reaction of the ozonide formed and the base can be suppressed, but at the same time sufficiently high conversions for the two component reactions can still be maintained.

The allyl alcohol employed in a process according to the invention, in particular by one of the preferred process embodiments described above, preferably has the general formula (Ia)

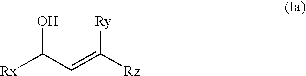

wherein Rx denotes an organic radical, and wherein furthermore Ry and Rz independently of one another can denote hydrogen or substituted or unsubstituted alkyl, alkenyl, cycloalkyl or aryl and the two radicals together can form a ring and/or one or both of the radicals Ry and Rz can form a ring together with the radical Rx.

It is particularly preferable for Rx to denote an organic radical, preferably an organic radical having at most 50 carbon atoms.

Ry and Rz independently of one another can denote hydrogen or substituted or unsubstituted alkyl, alkenyl, cycloalkyl, heterocycloalkyl, cycloalkylalkylene or (hetero)aryl,
or
Ry and Rz together can form a ring, preferably a ring having 5 to 20 members in total,
or
one or both of the radicals Ry and Rz can form a ring together with the radical Rx, preferably a ring having 5 to 20 members in total.

The process according to the invention can be carried out with a large number of secondary allyl alcohols of very different structure.

Ry and Rz independently of one another preferably denote hydrogen or substituted or unsubstituted straight- or branched-chain C₁-C₂₀-alkyl, straight- or branched-chain C₃-C₂₀-alkenyl, C₃-C₂₀-cycloalkyl, C₃-C₂₀-heterocycloalkyl, C₃-C₂₀-cycloalkylalkylene or C₅-C₂₀-(hetero)aryl.

In preferred alcohols of the formula (Ia), Rx is chosen from organic radicals having up to 30 carbon atoms and up to 10 nitrogen and/or oxygen atoms.

Particularly preferably, Rx denotes substituted or unsubstituted straight- or branched-chain C₁-C₂₀-alkyl, straight- or branched-chain C₅-C₂₀-alkenyl, C₃-C₂₀-cycloalkyl, C₃-C₂₀-heterocycloalkyl, C₃-C₂₀-cycloalkylalkylene or C₅-C₂₀-(hetero)aryl.

If Rx, Ry and/or Rz are a substituted alkyl, alkenyl, cycloalkyl, heterocycloalkyl, cycloalkylalkylene or (hetero)aryl, in each case the following substituents are preferred:

hydroxyl,
C₁-C₈-alkyl, preferably methyl, ethyl, n-propyl, iso-propyl, n-butyl, iso-butyl, tert-butyl,
C₃-C₁₈-cycloalkyl, preferably cyclopropyl, cyclopentyl, cyclohexyl, cyclooctyl, cyclododecyl, cyclopentadecyl, cyclohexadecyl,
C₂-C₈-alkynyl, preferably ethynyl, propynyl
C₁-C₈-perfluoroalkyl, preferably trifluoromethyl,
C₁-C4-alkoxy, preferably methoxy, ethoxy, iso-propoxy, n-butoxy, iso-butoxy, tert-butoxy,
C₃-C₁₂-cycloalkoxy, preferably C₃₋cycloalkoxy, C₅-cycloalkoxy, C₆₋cycloalkoxy, C₈-cycloalkoxy, C₁₂-cycloalkoxy, C₁₅-cycloalkoxy, C₁₆-cycloalkoxy,
C₁-C₂₀-alkoxyalkyl, in which 1 to 5 CH₂ groups are replaced by oxygen, preferably —[—O—CH₂—CH₂—]ₙ-Q or -[—O—CH₂—CHMe-]ₙ-Q, wherein Q is OH or CH₃ and wherein n can denote 1 to 4,
C₁-C₄-acyl, preferably acetyl,
C₁-C₄-carboxy, preferably CO₂Me, CO₂Et, CO₂i-Pr, CO₂^{t}Bu,
C₁-C₄-acyloxy, preferably acetyloxy,
halide, preferably F or Cl, and
Si₁—Si₃₀-siloxy.

The good results are obtained in particular if the double bond in the alpha position is not part of a system of conjugated double bonds. Preferably, the radicals Ry and Rz independently of one another are therefore hydrogen or alkyl, and Ry and Rz particularly preferably denote hydrogen.

If the alcohol to be reacted carries further alcohol groups or other groups which are not be reacted, in addition to an alcohol group which is to be reacted, these functionalities are expediently protected against ozonolysis.

Derivatization of the compounds serves in particular to introduce protective groups for non-allylic double bonds (double bonds which are not in the alpha position) optionally present, preferably by selective epoxidation thereof.

The invention is explained in more detail in the following with the aid of embodiment examples.

### General Instructions for the Reaction Procedure

The reactions were carried out in conventional laboratory apparatuses. In smaller batches, the reaction mixtures were kept at the appropriate temperature by dry ice baths. In the case of larger batches, double-jacketed vessels through which a cooling medium suitable for the desired temperature range was pumped were used.

Non-allylic double bonds present in the compounds were as a rule protected from ozonolysis by epoxidation.

Unless stated otherwise, all data in % are to be understood as % by weight data. Amounts data in the examples relate to weight ratios. Room temperature corresponds to about 20° C.

### EXAMPLE 1

### Reaction of oct-1-en-3-ol (VIII) with Ozone to Give Hexanal (IX)

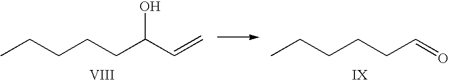

38.4 g (0.3 mol) oct-1-en-3-ol are initially introduced into 218 g toluene. Ozone-containing oxygen (32 g/h, 150 l, ozone content: 11 vol. %) is passed into the reaction mixture at −5° C. in the course of 2 h. 24 g (0.6 mol) sodium hydroxide in 456 g water are added dropwise to the reaction mixture over the entire reaction time. After the excess ozone has been driven off, the reaction mixture is warmed and is stirred at room temperature for 3 h. The organic phase is then separated off, and this is washed neutral and free from peroxide and finally concentrated. Conversion of octenol: 90.8% (GC-MS).

For better content assay, the hexanal formed is converted into the corresponding propylene glycol acetal in toluene solution with 24 g (0.31 mol) propylene glycol with 1 mol % para-toluenesulfonic acid. It was possible to determine the formation of the hexanal propylene glycol acetal by GC-MS.

Yield of hexanal propylene glycol acetal: 29.2 g, corresponding to 61.5% of theory, based on (IX)

### EXAMPLE 2

### Ozonolysis of 4-methyltridec-1-en-3-ol (X) to Give 2-methylundecanal

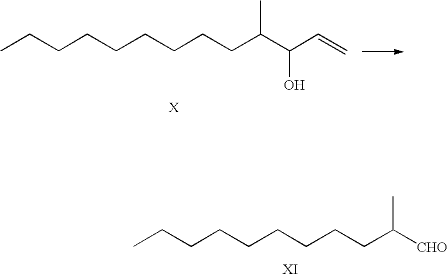

### EXAMPLE 2.1

### Preparation of the Secondary Ally Alcohol (X)

9.0 g (0.045 mol) 2-methylundecanal are initially introduced into 100 g tetrahydrofuran and the solution is cooled to −25° C. Vinylmagnesium bromide solution (0.05 mol, 50 ml of a 1 M solution in tetrahydrofuran) is added dropwise to the solution at this temperature, while stirring. After the addition, the reaction mixture is warmed to room temperature and heated under reflux for 2 h. The reaction mixture is then stirred at room temperature for a further 5 h and subsequently hydrolyzed with 20 g water. After the evolution of heat has subsided, the precipitate formed is dissolved with 5% strength hydrochloric acid. After the aqueous phase has been separated off, 200 g diethyl ether are added to the reaction mixture and the mixture is washed with 200 g water for neutralization. After the solvent has been distilled off, the crude product is obtained as a pale yellow oil.

Crude yield: 10.6 g, content: 90% (GC-MS).

### EXAMPLE 2.2

### Ozonolysis of (X) to Give Methylundecanal (XI)

The product from Example 2.1 is taken up in 100 g toluene and the reaction mixture is cooled to −5° C. The reaction solution is then gassed with ozone-containing oxygen (10 vol. % ozone in oxygen) over a period of 2 h. During the entire reaction time, 1.5 molar equivalents of sodium hydroxide, based on the allyl alcohol, in the form of a 10% strength solution in water are added dropwise to the reaction mixture. After the ozone which has not been consumed has been gassed out, the organic phase is separated off and washed neutral. After any peroxides still present have been destroyed, the solvent is separated off by distillation in vacuo, Conversion of secondary allyl alcohols 100%

Crude yield: 9.67 g, content: 85.6% (GC-MS) corresponding to 80% of theory, based on (X)

### EXAMPLE 3

### Ozonolysis of 1-phenylprop-2-enol (XII) to Give Benzaldehyde (XIII)

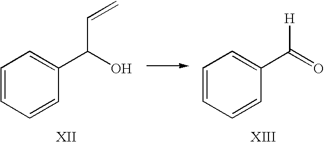

### EXAMPLE 3.1

### Preparation of Phenylprop-2-enol (XII)

5.0 g (0.05 mol) benzaldehyde are dissolved in 50 g tetrahydrofuran and the solution is cooled to −20° C. Vinylmagnesium bromide is added dropwise to this cooled solution as a 1 molar solution in tetrahydrofuran (1.05 molar equivalents, based on the benzaldehyde). After the dropwise addition, the solution is warmed to room temperature and stirred at room temperature for a further 5 h, 100 g water are then added to the reaction solution for hydrolysis. After the precipitate has been dissolved with 5% strength hydrochloric acid, the reaction solution is washed neutral and the organic phase is freed from the solvent in vacuo.

Crude yield: 6.0 g, content. 91% (GC-MS).

### EXAMPLE 3.2

### Ozonoysis of Phenylprop-2-enol (XII) to Give Benzaldehyde (XIII)

The secondary allyl alcohol (XII) from Example 3.1 is dissolved in 120 g toluene and the solution is subjected to ozonolysis at −5° C. During the entire duration of the ozonolysis, an equimolar amount (based on the secondary allyl alcohol (XII)) of NaOH is added dropwise as a 5% strength solution in water. After the end of the addition of ozone, the reaction mixture is degassed and warmed to room temperature. After the aqueous phase has been separated off, the organic phase is washed neutral and free from peroxide and the reaction mixture is freed from the solvent in vacuo.

Crude yield: 3.88 g, content, 90% (GC-MS), corresponding to 80% of theory

### EXAMPLE 4

### Ozonolysis of 1-cyclohexylprop-2-enol(XIV) to Give Cyclohexylcarbaldehyde (XV)

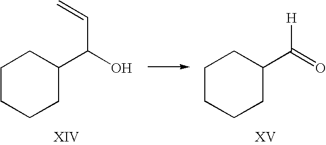

### EXAMPLE 4.1

### Preparation of Cyclohexylprop-2-enol (XIV) from Acrolein and Cyclohexyl Bromide

2.4 g (0.1 mol) Mg filings are initially introduced into diethyl ether (50 g). After activation of the magnesium with iodine, 16.3 g (0.1 mol) cyclohexyl bromide are added dropwise such that the reaction mixture boils gently. When the addition has ended, the mixture is heated under reflux for a further 2 h, until the magnesium has been consumed completely. 5.9 g (0.11 mol) acrolein are then added dropwise to the solution, which has been cooled to room temperature, and the reaction mixture is heated under reflux for a further 1.5 h to bring the conversion to completion. Working up after the hydrolysis with 100 g water comprises dissolving the precipitate, separating off the organic phase and washing the organic phase neutral, as described in Example 3.1.

Crude yield: 10.7 g, content: 71% (GC-MS)

### EXAMPLE 4.2

### Ozonolysis of Cyclohexylpropen-2-ol (XIV) to Give Cyclohexylcarbaldehyde (XV)

Analogously to Example 3.2, 1.2 g of the secondary allyl alcohol (XIV) from Example 4.1 are dissolved in twenty times the weight of toluene and subjected to ozonolysis at −5° C. and the product is worked up.

Crude yield: 0.9 g

Conversion of secondary allyl alcohol: 100%, aldehyde selectivity: 37.9%

### EXAMPLE 5

### Preparation of Nonal (XVII) by Ozonolysis of Undec-1-en-3-ol (XVI)

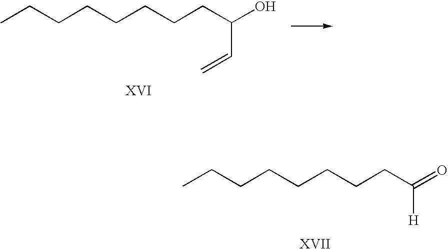

### EXAMPLE 5.1

### Reaction of Octyl Bromide with Acrolein to Give (XVI)

The preparation of undec-1-en-3-ol (XVI) is carried out using 19.3 g (0.10 mol) octyl bromide and the corresponding molar amounts of magnesium and acrolein according to Example 4.1.

Crude yield: 13.8 g, content: 80% (GC-MS).

### EXAMPLE 5.2

### Ozonolysis for the Preparation of Nonanal

The preparation of nonanal (XVII) from the secondary allyl alcohol (XVI) of Example 5.1 is carried out analogously to Example 3.2. After working up, nonanal (XVII) is obtained as a yellowish oil.

Conversion of secondary allyl alcohol: 24.3%, aldehyde selectivity: 100%

### EXAMPLE 6

### Ozonolysis of 3,5,5-trimethyl-cyclohex-2-enol XVIII) to give 3,3-dimethyl-5-oxohexanal (XIX)

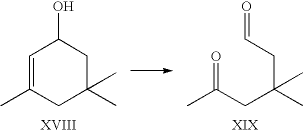

28 g (0.2 mol) of the secondary cyclic allyl alcohol (XVIII) are dissolved in 120 g toluene and the solution is subjected to ozonolysis at −10° C. During the entire duration of the ozonolysis (about 1.5 h), 1.5 molar equivalents, based on the secondary allyl alcohol (XVIII), of NaOH are added dropwise as a 10% strength solution in water. After the end of the addition of ozone, the reaction mixture is degassed and warmed to room temperature. After the aqueous phase has been separated off, the organic phase is washed neutral and free from peroxide and the reaction mixture is freed from the solvent in vacuo.

Crude yield: 24.6 g (50% of theory)

Conversion of allyl alcohol=100%, aldehyde selectivity ˜50% (GC-MS)

1-8. (canceled)

9. A process for preparing an aldehyde from a secondary allyl alcohol, comprising:
a) providing an alcohol;
b) treating said alcohol with ozone.

10. The process of claim 9, wherein said alcohol is treated with 1-3 molar equivalents of ozone per alcohol group to be treated.

11. The process of claim 9, wherein in b) an inorganic base is added.

12. The process of claim 11, wherein said inorganic base is selected from the group consisting of NaOH, KOH, LiOH, NaHCO₃, Na₂CO₃, CaCO₃, and mixtures thereof.

13. The process of claim 11, wherein said inorganic base is employed in an aqueous solvent.

14. The process of claim 9, wherein said alcohol is provided in an organic solvent.

15. The process of claim 9, wherein the reaction in b) takes place at a temperature in the range of from −78° C. to +30° C.

16. The process of claim 9, wherein said alcohol has the general formula (Ia): 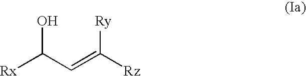
wherein Rx is an organic radical, and Ry and Rz, independently of one another, are hydrogen or substituted or unsubstituted alkyl, alkenyl, cycloalkyl, or aryl, wherein Ry and Rz together define a ring system and/or one or both of Ry and Rz define a ring system together with Rx.
